(19) United States
(12) Patent Application Publication
(10) Pub. No.: US 2018/0345031 A1
(43) Pub. Date: Dec. 6, 2018

(54) WEARABLE CAP FOR CLINICAL TRANSCRANIAL MAGNETIC STIMULATION (TMS) TREATMENT OF DEPRESSION: An aid for clinician localization & determination of treatment area

(71) Applicant: Alexis Meshi, Newport Beach, CA (US)

(72) Inventor: Alexis Meshi, Newport Beach, CA (US)

(21) Appl. No.: 15/613,067

(22) Filed: Jun. 2, 2017

(51) Int. Cl. A61N 2/00; A61N 2/02; A61N 1/05

(52) U.S. Cl. CPC A61N 2/006; A61N 1/0526; A61N 2/02

(57) ABSTRACT

A cost effective new method to help a clinician (Physician) quickly and accurately locate and determine the treatment area for providing Transcranial Magnetic Stimulation (TMS) to a patient struggling with a psychiatric illness (i.e.: Depression).

### CROSS REFERENCE

Provisional Application #62/508,969

### BACKGROUND

US Patent Classification: A61B5/6803, A61N1/0526, A61N1/36082, A61N2/006

Research has shown that TMS treatment works well for the treatment of Depression. The FDA cleared TMS in the United States as a treatment for depression in 2008. There are currently five FDA approved systems. Four of them have a figure 8 coil and one has a Hesed (or “H”) coil. The coils are used to administer the electromagnetic pulses of the system to the patient's head for treatment.

Treatment by a clinician (Psychiatrist, MD) is administered at the LDLPFC (LEFT dorso-lateral prefrontal cortex). Engineers define the Figure 8 coil “target” as being 2 cm×2 cm, to 2 cm×3 cm. The Hesed Coil has broader coverage with a target >3-4 cm in diameter.

Studies with focal coils, have looked at efficacy. The effectiveness of the treatment. Efficacy seems to be dependent on the stimulation site and on the accuracy of the methods to locate the target of stimulation.

Prior to administering treatment the clinician (Psychiatrist, MD) has to locate the motor cortex, determine the Motor Threshold and then locate the LDLPFC to proceed with treatment. This is known as “Prescription Dosing”.

Location Mapping and Motor Threshold determination are important for neurophysiological integrity of treatment and for the psychological integrity of the patient. The clinician must be able to demonstrate knowledge and competence in the localization and determination process. This affects the care given to the patient and the outcome of treatment.

There are currently 6 ways/methods to locate these areas for clinical treatment. Through research and refinement the most accurate thus far is the Modified BEAM/F3 method. These methods have included: 1) 5 cm, 6 cm, and 7 cm rule 2) International 10:20 System (which takes into account skull sizes) 3) Universal EEG Caps 4) BEAM Method 5) Neuro-Navigational Equipment 6) Modified BEAM/F3 method.

The problem with the initial 5 cm rule (physically measuring 5 cm forward, on the patient's head, from the motor cortex to the LDLPFC) that earlier studies (O'Reardon 2007) found was that it was overall inaccurate and ineffective. It was subsequently noted that a “more lateral and anterior approach” would be more effective (Herbsman, et al 2009; Ahdab, et al. 2012). However this continued to use a very time consuming, non-consistent and somewhat sloppy physical approach of measuring this distance directly on the patient's head.

It was eventually found that both the 5 & 6 cm rules were insufficient for placement based on a 2014 study (Lefaucher et al 2014) that eventually recommended that a more accurate location would be closer to 7 cm or F3 (of the international 10:20 system). The international 10:20 system takes into account the “skull size” of the individual patient. The Left Dorsal Lateral Prefrontal Cortex corresponds to F3. However, like the previous method this system continues to have the Physician use a tape measure and do a series of calculations to find the appropriate position on the patient's head and mark them with a sharpie pen or the like. (See Drawing #1)

Universal EEG Caps were then found to be helpful in the localization process. They do take into account varied head sizes. They are not intended for use with treating clinical depression or Psychiatric uses. They have been used for Neurological utility. They are expensive and non-hygienic (they do not cover the hair of patients, the coil comes into contact with the patient's head/hair). They cannot be used for multiple treatments without washing them. (See Drawing #2)

The Beam Method is relatively inexpensive to patient and provider but requires training time and again requires the use of a tape measure, computer (software to do calculations), dry erase marker and optional use of a cap. (Beam, Borchardt, Reeves & George 2009) (See Drawing #3)

Neuro Navigational equipment while available at academic research centers for use is not available in the average community psychiatric clinic. They are also very expensive ranging from several hundred thousand dollars to several millions of dollars. Studies thus far have not shown them to be more accurate than other methods (Blumberger et al 2016). (See Drawing #4)

The Modified Beam method is theoretically the most accurate if done correctly. This relies on the clinician ability. It modifies the Beam method slightly. But still relies on the clinician making multiple measurements using a measuring tape on the patient's head and marking locations with a sharpie marker or the like. The clinician enters these measurements into a software system and can supposedly accurately determine the MT determination area and the treatment (LDLPFC/F3) area.

These methods are currently considered the Gold Standard and taught by leaders and members of the Clinical TMS Society in educating general community psychiatrists in using TMS to treat their patients. In fact, it was when I attended the 5^{th} Annual Clinical TMS Society Meeting's Inaugural Workshop in San Diego in May 2017 that I thought of this invention.

### BRIEF SUMMARY OF THE INVENTION

With the “Meshi Cap”, time can be saved and cost efficiency and treatment consistency achieved for clinicians. Appropriate hygiene can be achieved and patient comfort, confidence and therefore treatment outcomes can also be significantly improved.

The “Meshi Cap” is a new method for localization of treatment areas for TMS treatment of psychiatric diagnoses including depression.

### DETAILED DESCRIPTION OF THE INVENTION

The invention is a cloth cap with a stretchable under the chin non-metallic adjustable cloth strap (Velcro), the chin strap has an opening at the bottom to allow the chin to go through it. The cap has ear holes. There will be an adjustable & stretchable cloth back strap from the inion (back of the cap) to the chin strap (Velcro). The front part of the cap will sit just above the nasion (the flat area just above the nose) and the back part of the cap will be placed over the inion (the bony prominence at the base of the skull). The cap will sit just above the ears. It comes in 4 sizes x-small (45.5-49 cm head circumference), small (49.5-54 cm head circumference), medium (54.5-57 cm), and large (57.5-62 cm head circumference) and is pre-marked with a portion of the 10:20 system to treat a particular psychiatric diagnosis or location of the brain.

This particular product/method will be used for depression. So the cap will be marked in the locations for Cz or Vertex, C3 and F3. The cap will be marked for these areas with a “target”. Next to the targets will be an instruction or “call to action” for the clinician, i.e.: MT (for motor threshold determination) or “treat” for the LDLPFC. (See FIGS. #1 & #2)

1) A New Method for the clinician (especially a Psychiatrist in community clinical practice) to locate the motor cortex and treatment area of a patient's head/brain to provide Transcranial Magnetic Stimulation (TMS) treatment for a neurological, pain or psychiatric diagnosis.

2) A New Method for the clinician (especially a Psychiatrist in community clinical practice) to locate the motor cortex and the LDLPFC of a patient's head/brain to provide/administer Transcranial Magnetic Stimulation (TMS) Treatment for Depression.

3) The “Meshi Cap” allows for time reduction for the clinician.

4) It also allows for cost reduction for the clinician.

5) It allows for better consistency between clinicians (who may be trying to determine treatment locations) and improves consistency between treatment sessions for the same clinician.

6) It improves hygiene for the patient undergoing TMS treatment.

7) It gives a much more smooth and polished look and TMS experience to both the clinician and the patient.

8) Lastly, it provides for greater patient comfort and confidence in the treatment which can affect treatment adherence/compliance and outcome.
